(19) United States
(12) Patent Application Publication
(10) Pub. No.: US 2013/0255361 A1
(43) Pub. Date: Oct. 3, 2013

(54) Methods and Devices for Multi-Dimensional Separation, Isolation and Characterization of Circulating Tumour Cells

(71) Applicant: The Royal Institution for the Advancement of Learning / McGill University, (US)

(72) Inventors: David Juncker, Verdun (CA); Nicholas Bertos, Cote St. Luc (CA)

(73) Assignee: The Royal Institution for the Advancement of Learning / McGill University, Montreal (CA)

(21) Appl. No.: 13/850,596

(22) Filed: Mar. 26, 2013

Related U.S. Application Data
(60) Provisional application No. 61/617,714, filed on Mar. 30, 2012.

(51) Int. Cl. C12M 1/12; G01N 1/10

(52) U.S. Cl. CPC C12M 1/12; G01N 1/10
USPC 73/61.59; 210/335

(57) ABSTRACT

In most cancers, a significant factor in a poor outcome for the individual cancer victim is metastatic disease, i.e., dissemination of tumour cells to other parts of the human body via the circulation, such as distant organs, and their subsequent proliferation therein to form multiple other cancer tumours. The presence of circulating tumour cells, or CTCs, represents a vital intermediate step in this process and variations of a few CTCs within blood samples containing tens of billions of cells may denote the outcome for a patient or impact the cancer treatment regimen. At present no low cost field deployable technique for filtering CTCs exists. According to embodiments of the invention micro-machined filters with high aspect ratio, with and without, functionalization are employed to perform multi-parameter filtering for CTCs based upon compatibility with low cost semiconductor processes within multiple materials including silicon, polymers and silicon carbide.

### CROSS REFERENCE TO RELATED APPLICATIONS

This patent application claims the benefit of U.S. Provisional Patent Application U.S. 61/617,714 filed Mar. 30, 2012 entitled “Methods and Systems for Multi-Dimensional Separation, Isolation and Characterization of Circulating Tumour Cells”, the entire contents of which are included by reference.

### FIELD OF THE INVENTION

This invention relates to micro-machined filters and more specifically high aspect ratio micro-machined filters for use in separation and isolation of circulating tumour cells.

### BACKGROUND OF THE INVENTION

Cancer, known medically as a malignant neoplasm, is a term for a large group of different diseases, all involving unregulated cell growth. In cancer, cells divide and grow uncontrollably, forming malignant tumors, and can invade nearby parts of the body, and may also spread to more distant parts of the body through the lymphatic system or bloodstream. However, not all tumors are cancerous. Those referred to as benign tumors do not grow uncontrollably, do not invade neighbouring tissues, and do not spread throughout the body.

Healthy cells control their own growth and will destroy themselves if they become unhealthy. Cell division is a complex process that is normally tightly regulated. Cancer occurs when problems in the genes of a cell, or other causes, prevent these controls from functioning properly. These problems may come from damage to the gene or may be inherited, and can be caused by various sources inside or outside of the cell. Faults in two types of genes are especially important: oncogenes, which drive the growth of cancer cells, and tumor suppressor genes, which prevent cancer from developing. Determining what causes cancer is complex and it is often impossible to assign a specific cause for a specific cancer. Many things are known to increase the risk of cancer, including tobacco use, infection, radiation, lack of physical activity, poor diet and obesity, and environmental pollutants. These can directly damage genes or combine with existing genetic faults within cells to cause the disease. A small percentage of cancers, approximately five to ten percent, are entirely hereditary.

Cancer can be detected in a number of ways, including the presence of certain signs and symptoms, screening tests, or medical imaging. Once a possible cancer is detected it is typically diagnosed by microscopic examination of a tissue sample taken from the individual. Once diagnosed cancer is usually treated with chemotherapy, radiation therapy, surgery or a combination thereof. The chances of surviving the disease vary greatly according to the type and location of the cancer and the extent of the disease at the start of treatment. Accordingly, it is beneficial in all cancers to advance diagnosis to a stage as early in the development of the cancer as possible. Whilst cancer can affect people of all ages, and some types of cancer are more common in children, the risk of developing cancer generally increases with age. In 2007, cancer caused about 13% of all human deaths worldwide (7.9 million) and rates are rising as more people live to an old age and as mass lifestyle changes occur in the developing world.

In most cancers, a significant factor in a poor outcome for the individual cancer victim is metastatic disease, i.e., dissemination of tumour cells to other parts of the human body via the circulation, such as distant organs, and their subsequent proliferation therein to form multiple other cancer tumours. The presence of circulating tumour cells, or CTCs, represents a vital intermediate step in this process. The first report on CTCs dates back to 1869. Thomas Ashworth observed CTCs in the blood of a man with metastatic cancer, see “A case of cancer in which cells similar to those in the tumours were seen in the blood after death” (Aust. Med. J. 14, pp 146-149). However, recently interest in CTCs as predictive and prognostic markers, as well as in the context of basic research, has increased dramatically following technical advances permitting their isolation and quantitation. It has been demonstrated that the presence or persistence of CTCs in cancer is correlated with recurrence, poor outcome, and resistance to therapy, see for example C. Criscitiello et al in “Circulating tumor cells and emerging blood biomarkers in breast cancer” (Curr Opin Oncol 22, 552-8), B. P. Negin et al in “Circulating tumor cells in colorectal cancer: past, present, and future challenges” (Curr Treat Options Oncol 11, pp 1-13) and M. J. Serrano Fernadez, et al. in “Clinical relevance associated to the analysis of circulating tumour cells in patients with solid tumours” (Clin. Transl. Oncol. 11, pp 659-689). Thus, assays for CTCs now are emerging as a new element for the prediction of cancer outcome, as well as for potential early diagnosis and early prediction of response to therapy. However, at present these assays are laboratory-based research procedures.

There are multiple factors that influence the migration of research tests and protocols into routine screening tests within medical environments, such as clinics and hospitals, or consumer environments. These factors include, but are not limited to, the possible harm from performing the screening test, likelihood of correctly identifying cancer, likelihood of cancer being present, impact of follow-up procedures, availability of suitable treatment, does early detection improve treatment outcome, will the cancer ever need treatment, is the test acceptable to the patient, and the cost of the test. In respect of test costs some expert bodies, such as the U.S. Preventive Services Task Force (USPSTF), completely ignore the question of money. Most, however, include a cost-effectiveness analysis that, all else being equal, favors less expensive tests over more expensive tests, and attempt to balance the cost of the screening program against the benefits of using those funds for other health programs.

Accordingly, a test based upon CTCs should address these factors by being based upon blood samples they should have negligible impact on the majority of individuals and be manageable in individuals with haemophilia. Further CTC testing offers the potential for high sensitivity and high specificity thereby reducing false results as well as providing low cost multi-dimensional screening allowing routine testing of multiple cancers concurrently and may be tailored to demographic variances. Accordingly, a very low cost multi-dimensional testing of cancers my fundamentally adjusting screening and analysis decisions towards one of always testing.

At present the USPSTF strongly recommends cervical cancer screening in women who are sexually active and have a cervix at least until the age of 65. They also recommend colorectal cancer screening starting at age 50 until age 75. At present there is insufficient evidence to recommend for or against screening for skin cancer, oral cancer, lung cancer, or prostate cancer in men under 75. Routine screening is currently not recommended for bladder cancer, testicular cancer, ovarian cancer, pancreatic cancer, or prostate cancer. The USPSTF recommends mammography for breast cancer screening every two years for those 50-74 years old and does not recommend either breast self-examination or clinical breast examination.

Breast cancer remains the most frequent type of cancer in women, with approximately 212,600 new cases (1,300 male) diagnosed each year in the United States and 23,400 women in Canada. It is also among the leading causes of cancer death in women, representing 15% of all cancer deaths in women, with an estimated 40,200 cancer-related deaths (400 male) in 2003 in the United States (Surveillance, Epidemiology, and End Results Cancer Statistics Review, 1975-2000; http://seer.cancer.gov/csr/1975_{—}2000). The vast majority of these deaths are the result of a recurrent metastatic disease.

Despite years of clinical research, the odds of achieving complete response for patients with metastatic breast cancer (MBC) are extremely low, see for example M. Ellis et al in “Treatment of metastatic disease” (Diseases of the Breast, Second Edition, Lippincott-Raven, 2000, pp 749-799) and P. A. Greenberg in “Long-term follow-up of patients with complete remission following combination chemotherapy for metastatic breast cancer” (J Clin Oncol 14, pp 2197-2205, 1996). Only a few patients who achieve a complete response after chemotherapy remain in this state for prolonged periods of time, with some patients remaining in remission beyond 20 years, see Greenberg. These long-term survivors are usually young, have an excellent performance status, and, more importantly, have limited metastatic disease, see for example Y. Nieto et al “Phase II trial of high-dose chemotherapy with autologous stem cell transplant for stage IV breast cancer with minimal metastatic disease” (Clin Cancer Res 5, pp 1731-1737) and E. Rivera et al in “Fluorouracil, doxorubicin, and cyclophosphamide followed by tamoxifen as adjuvant treatment for patients with stage IV breast cancer who have no evidence of disease” (Breast J 8, pp 2-9). The majority of patients with metastatic disease respond transiently to conventional treatments and develop evidence of progressive disease within 12 to 24 months of initiating treatment see Ellis. For these patients, systemic treatment has not translated into a significant improvement in survival but has substantially improved their quality of life.

Circulating tumor cells (CTCs) can be detected in blood from patients with metastatic and primary carcinomas, see for example P. D. Beitsch in “Detection of carcinoma cells in the blood of breast cancer patients” (Am J Surg 180, pp 446-449), T. Fehm et al in “Cytogenetic evidence that circulating epithelial cells in patients with carcinoma are malignant” (Clin Cancer Res 8, pp 2073-2084), J. J. Gaforio in “Detection of breast cancer cells in the peripheral blood is positively correlated with estrogen-receptor status and predicts poor prognosis” (Int J Cancer 107, pp 984-990), and F. Austrup et al in “Prognostic value of genomic alterations in minimal residual cancer cells purified from the blood of breast cancer patients” (Br J Cancer 83, pp 1664-1673).

Over the past several years, the development of immunomagnetic platforms has permitted accurate enumeration of CTCs at extremely low frequencies, see L. Terstappen et al in “Peripheral blood tumor cell load reflects the clinical activity of the disease in patients with carcinoma of the breast” (Int J Oncol 17, pp:573-578). In several case reports, the presence of CTCs has been associated with shortened survival times, see for example R. W. Carey et al in “Carcinocythemia (carcinoma cell leukemia): An acute leukemia-like picture due to metastatic carcinoma cells” (Am J Med 60, pp 273-2′78). M. Cristofanilli et al in “Circulating Tumor Cells: A Novel Prognostic Factor for Newly Diagnosed Metastatic Breast Cancer” (J. Clin. One., Vol. 23 No. 7, pp 1420-1430 investigated whether the presence of circulating tumor cells (CTCs) predicts treatment efficacy, progression-free survival (PFS), and overall survival (OS) in patients with newly diagnosed MBC who were about to start first-line therapy.

In 2008 approximately 12.7 million cancers were diagnosed (excluding non-melanoma skin cancers and other non-invasive cancers) and 7.6 million people died of cancer worldwide, see A. Jemal et al in “Global cancer statistics” (CA: A Cancer Journal for Clinicians, Vol. 61 (2), pp 69-90). Accordingly, cancers as a group accounts for approximately 13% of all deaths each year with the most common being: lung cancer (1.3 million deaths), stomach cancer (803,000 deaths), colorectal cancer (639,000 deaths), liver cancer (610,000 deaths), and breast cancer (519,000 deaths). This makes invasive cancer the leading cause of death in the developed world and the second leading cause of death in the developing world with over half of cases occur in the developing world. 2011 cancer statistics reveal that over a lifetime, the probability for a Canadian of developing cancer is 50%, and the probability of dying from it is 25%.

Breast cancer is arguably the well-understood cancer owing to its high prevalence, and to the large research effort dedicated to understand and eradicate it, and it can serve as a model for other cancers and for pioneering new technologies. With breast cancer, a major challenge in treatment is posed by its heterogeneity. Breast cancer tumours are traditionally classified by immunohistochemical (IHC) tests on thin tumour sections, and more recently by gene expression profiles. These subdivisions are important because they predict both a patient's response to targeted therapies and a patient's prognosis, which informs decisions about systemic treatments. However, the accuracy of these tests remains limited, and in the case of a relapse following the development of metastatic disease, there is a dearth of molecular prognostic markers. Indeed, relapse is often assessed by radiology and therapeutic efficacy with assays against Mucin1 and carcinoembryonic (CEA) antigen, but the accuracy is limited. Despite these significant limitations, after decades of research and hundreds of millions of dollars of funding from Government and charity organizations for cancer research generally and the significant potential of CTCs, the clinical utility of monitoring CTC levels remains controversial, see W. J. Allard et al in “Tumor cells circulate in the peripheral blood of all major carcinomas but not in healthy subjects or patients with non-malignant diseases” (Clin Cancer Res 15, pp 6897-6904). Amongst the factors cited against CTCs are the diversity of CTCs, the likely involvement of circulating cancer stem cells (CCSC) and epithelial-to-mesenchymal transition cells (EMT-cells) in spreading cancer, and the technological challenges to isolate these rare cells rapidly and cost-effectively. Accordingly, it would be beneficial to provide a means of rapidly separating CTCs at low cost. It would be further beneficial if the separation could rapidly isolate CCSC and EMT-cells through multi-dimensional separation.

The potential of rapid low-cost CTC detection and analysis touches all levels of disease management, including early diagnosis, disease prognosis, selection of treatment based on CTC fingerprints, monitoring of therapeutic efficacy, and of recurrence of the disease. It would be evident that even if the CTC assays are successful in only a few of these areas, CTC detection could change the standard of care and improve outcome for many patients in a near future.

Challenges in CTC Isolation:

Studies of CTCs are hindered both by their rarity, and by an incomplete understanding of their overall features. Indeed the presence of as little as 1 CTC per 10 ml of blood may have prognostic significance, yet this volume also contains ˜10⁸ white blood cells with a size distribution overlapping that of CTCs, as well as ˜5×10¹⁰ erythrocytes and ˜5×10⁹ thrombocytes, both of which are significantly smaller but require removal. Accordingly, a viable CTC separation technology must achieve reproducible isolation of very small numbers of CTCs within overall cell quantities of approximately 6×10¹⁰, wherein differences of single CTCs can indicate significant prognoses, for example Cristofanilli reported that patients with ≧five CTCs at baseline and at first follow-up (4 weeks) had a worse prognosis than patients with less than five CTCs wherein both patient groups had newly diagnosed MBC. Within the prior art several techniques have been reported as outlined below.

EpCAM-Based CTC Isolation:

Epithelial cell adhesion molecule (EpCAM) is a protein that in humans is encoded by the EPCAM gene. EpCAM is a pan-epithelial differentiation antigen that is expressed on almost all carcinomas. Most traditional enrichment and enumeration methods including manual enumeration on slides, flow cytometry, and centrifugation, are based on the definition of CTCs as nucleated cells bearing epithelial markers, such as EpCAM (epithelial cell adhesion molecule) or epithelial-specific cytokeratins, and lacking expression of hematopoietic-lineage markers, e.g. protein tyrosine phosphatase, receptor type, C also known as PTPRC or CD45. For example, the U.S. Food and Drug Administration approved Veridex CellSearch platform identifies CTCs by positive staining for both 4′,6-diamidino-2-phenylindole (DAPI) and anti-pan-cytokeratin and negative staining for CD45 following an anti-EpCAM-antibody-based affinity purification step. This system has been widely used for many studies. Recently, microfluidics isolation techniques using posts, see for example S. Nagrath, et al. “Isolation of rare circulating tumour cells in cancer patients by microchip technology” (Nature 450, pp 1235-1239), and vortices, see S. L. Stott, et al in “Isolation of circulating tumor cells using a microvortex-generating herringbone-chip” (PNAS 107, pp 18392-18397) were to be superior to the Veridex CellSearch system in detecting CTCs, albeit at the cost of long processing times up to 10 hours per sample.

Limitation of EpCAM Based Isolation:

The sensitivity of EpCAM suffers from practical and biological limitations. The biological limitation stems from the fact that there is mounting evidence of heterogeneity among CTCs. For example, ˜10% of breast tumours fail to express EpCAM, and within EpCAM-positive primary tumours, EpCAM expression is heterogeneous, suggesting that there exists a set of EpCAM-negative CTCs, which will be missed using EpCAM-based technologies—such cells have indeed been identified in metastatic breast cancer. Additionally, EpCAM expression within primary breast tumours is correlated with poor outcome in node-positive disease; thus, the prognostic value of CTCs detected by solely EpCAM-based isolation methods is likely conflated with that of lymph node positivity.

Parallel studies conducted using EpCAM and cell size-based CTC isolation technologies reveal that each method identified different, although partially overlapping subsets of patients as CTC-positive, and that patients identified as CTC-positive by both methods had worse outcomes than those identified as CTC positive by either method alone. Interestingly, CTCs could be isolated in patients who had not experienced disease recurrence 7-22 years after curative surgery, further suggesting that CTCs comprise a heterogeneous population and that in fact only specific subsets of CTCs are linked to disease recurrence. In addition, cells that have undergone an epithelial-to-mesenchymal transition (EMT) as well as circulating cancer stem cells (CCSC) likely lack the markers (e.g. EpCAM) used for CTC isolation, yet could be the cells responsible for disseminating the disease (see below for more details).

Non-EpCAM-Based Microfluidic Isolation:

In addition to EpCAM methodologies there have been widespread and creative efforts to develop CTC isolation technologies using many different approaches such as mechanical filtration based on size and rigidity, inertial filtration, as well as continuous and pulsed deterministic ratchets, see for example Q. Guo et al in “Deterministic microfluidic ratchet based on the deformation of individual cells” (Physical Review E 83, pp. 2731-2737). However, many of these demonstrations are at an early stage, have been limited to specific culture cell lines such as MCF7 (a breast cancer cell line—Michigan Cancer Foundation—7), and MDA-MB-231 (another human breast cancer cell line), that were spiked into serum, and lack the required throughput for rapidly screening 10 ml of blood, which represents the typical sample volume.

Filtration Based CTC Isolation:

The potential of filtration for CTC isolation was recognized as early as 1958, but was limited by the availability of filtration membranes with well-defined pore sizes, see L. Long et al in “Simplified technique for separation of cancer cells from blood” (J. American Med. Assoc. 170, pp 1785-1788). Set-ups with track-etched membranes with 8 μm holes etched vertically into the membrane following synchrotron radiation and randomly distributed across the surface have been successfully used for CTC enrichment, see H. J. Kahn, et al “Enumeration of Circulating Tumor Cells in the Blood of Breast Cancer Patients After Filtration Enrichment: Correlation with Disease Stage” (Breast Cancer Res. and Treatment 86, pp 237-24′7). However, only a low density of holes is attainable to avoid coalescence of holes, entailing high flow resistance and the difficulty to process raw blood. This setup often requires centrifugation to isolate red blood cells, making it cumbersome for clinical use, and complicating the required regulatory approval.

Micro-Fabricated Parylene Filters:

An important advance was the introduction of micro-fabricated membranes etched into 10 μm-thick parylene-C, a variety of chemical vapor deposited polyp-xylylene) polymer used for coating printed circuit boards (PCBs) and medical devices. Using these, successful isolation of CTCs in the blood of diverse cancer patients with higher yield than CellSearch and with the ease needed for clinical translation was achieved, see H. K. Lin et al “Portable Filter-Based Microdevice for Detection and Characterization of Circulating Tumor Cells” (Clinical Cancer Res. 16, pp 5011-5018) and S. Zheng et al in “3D micro-filter device for viable circulating tumor cell (CTC) enrichment from blood” (Biomed. Microdevices 13, pp 203-213). In addition, whereas early methods were based on fixing the cells, see Lin, the technique has also been demonstrated with live cells, see Zheng. An important limitation of this technique, however, is the auto fluorescence of parylene. Furthermore, parylene is inert, which is why it is used in coating medical devices, and accordingly it is difficult to functionalize it with other inert coatings or biomolecules for example. In addition, purely size-based approaches will by definition yield an enriched population of cells that are larger than normal blood cells. However, tumour cells are heterogeneous in size, and many CTCs are likely missed.

Challenges to Clinical Translation of CTCs:

The clinical translation of CTCs has not been realized yet, see Criscitiello and M. S. Wicha et al in “Circulating Tumor Cells: Not All Detected Cells Are Bad and Not All Bad Cells Are Detected” (J. Clin. Oncology, Vol 29(12), pp 1508-11). Accordingly, the challenges associated with reliably isolating CTCs at affordable cost constitute a major impediment. Further, the mounting evidence of the heterogeneity of CTCs has not been fully addressed with current isolation techniques which are generally based on a single parameter, either a physical property or the expression of one or two specific proteins, and thus are predicted to fail to isolate CTCs falling outside of those matching this predetermined criterion. Furthermore, it is becoming clear that EMT cells play an important role in cancer progression, see for example K. Polyak et al in “Transitions between epithelial and mesenchymal states: Acquisition of malignant and stem cell traits” (Nat Rev Cancer 9, pp 265-73). EMT in tumour cells is linked to invasion, metastasis and the acquisition of resistance to therapy, and thus forms an important area for research and potential clinical intervention.

EMT has also been linked to cancer stem cells, which have been proposed to be the source of metastatic lesions, and markers for both processes have been observed to be present in CTCs. Thus, the ability to isolate CTCs based on multiple markers, including cell surface EMT markers (e.g., N-cadherin) and stem cell markers (e.g., CD44+/CD24− status or CD133, these being glycoproteins) in conjunction with selection against non-CTC extracellular epitopes will support high-throughput patient screening for these features. In addition, CTCs were for example found as clusters in animal models and shown to more successfully metastasize than individual cells, see for example L. A. Liotta et al in “Quantitative Relationships of Intravascular Tumor Cells, Tumor Vessels, and Pulmonary Metastases following Tumor Implantation” (Cancer Research 34, pp 997-1004), and clusters have recently also been found in cancer patients, see for example T. E. Witzig, et al “Detection of Circulating Cytokeratin-positive Cells in the Blood of Breast Cancer Patients Using Immunomagnetic Enrichment and Digital Microscopy” (Clinical Cancer Research 8, pp 1085-1091) yet current technologies generally fail to isolate aggregates.

Requirements for CTC Isolation Technologies:

For clinical use, isolation technologies should be fast, sensitive and selective, while they should also capture the full gamut of CTCs, including CCSC and EMT cells. Among the technologies developed to date, only filter-based approaches are fast and sensitive, but none can simultaneously target multiple characteristics. While some cancers, e.g. breast cancers, have been classified based on pathophysiological features, which is also reflected in their molecular fingerprints, it is unclear precisely how this affects the rate of CTC positivity and the molecular characteristics of CTCs or of EMT-cells and CCSC. Since heterogeneity among and within patients has already been established, previous methods based on a single selection criterion are not well suited to uncover the heterogeneity of circulating cells that likely exist within a single patient, see Wicha. Accordingly, it would be beneficial to provide a technology solution that can isolate a range of circulating cells based on diverse characteristics, both mechanical and molecular, thereby allowing CTCs, CCSCs and EMT-cells to be detected, differentiated and classified. Such a technology with a low cost and fast analysis would allow improvements in cancer management, including diagnosis, prognosis, therapeutic decision-making, assessment of treatment efficacy and monitoring of recurrence.

The inventors have developed multi-stage micro-fabricated Si filters with hole dimensions from 20 μm down to 6 μm, as well as filters coated with antibodies against cancer cell membrane proteins, together with a cartridge system allowing multiple filter stacks to be assembled with low cost in multiple filter configurations. The inventors have further established novel polymer fabrication technologies for low cost mass production of elastomeric polymer membranes.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate drawbacks within the prior art relating to micro-machined filters and more specifically to high aspect ratio micro-machined filters for use in separation and isolation of circulating tumour cells.

In accordance with an embodiment of the invention there is provided a method comprising providing at least one micro-fabricated filter of a plurality of micro-fabricated filters, each micro-fabricated filter comprising holes of predetermined geometry and predetermined dimensions for capturing a predetermined cell type and assembling the at least one micro-fabricated filter of the plurality of micro-fabricated filters within a housing. The method further comprising exposing the at least one micro-fabricated filter of a plurality of micro-fabricated filters to a fluid and measuring the at least one micro-fabricated filter of a plurality of micro-fabricated filters to determine the presence of the predetermined cell type.

In accordance with an embodiment of the invention there is provided a method comprising providing a series of micro-fabricated filters representing a predetermined subset of a plurality of micro-fabricated filters, each micro-fabricated filter comprising holes of predetermined geometry and predetermined dimensions for capturing a predetermined cell type, and assembling the series of micro-fabricated filters in a predetermined order based upon the plurality of predetermined cell types.

In accordance with an embodiment of the invention there is provided a method comprising a series of micro-fabricated filters representing a predetermined subset of a plurality of micro-fabricated filters, each micro-fabricated filter comprising holes of predetermined geometry and predetermined dimensions for capturing a predetermined cell type, wherein the series of micro-fabricated filters are disposed in a predetermined order for sequentially filtering a fluid passing through them based upon the plurality of predetermined cell types.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts methods of CTC isolation according to the prior art;

FIG. 2 depicts a method of CTC isolation by immunomagnetic separation according to the prior art;

FIG. 3 depicts a method of CTC isolation through antibody coated surfaces according to the prior art;

FIG. 4 depicts a method of CTC isolation through nano-patterned surfaces to enhance cell capture according to the prior art;

FIG. 5 depicts a method of CTC isolation through antibody coated surfaces with wavy channels to increase capture surface area according to the prior art;

FIG. 6 depicts a method of CTC isolation through antibody coated surfaces with rapid microvortex flow according to the prior art;

FIG. 7 depicts CTC isolation according to an embodiment of the invention allowing multi-parameter isolation;

FIG. 8 depicts a micro-machined silicon filter for CTC isolation according to an embodiment of the invention;

FIG. 9 depicts a stackable modular filter assembly for CTC isolation according to an embodiment of the invention;

FIG. 10 depicts optical micrographs of Vybrant™ fluorescent dye-labelled MCF7 cells filtered using micro-machined silicon filters according to an embodiment of the invention;

FIG. 11 depicts optical visualizations of CTC cells filtered using a micro-machined silicon filter according to an embodiment of the invention;

FIG. 12 depicts a stackable modular filter assembly for CTC isolation according to an embodiment of the invention;

FIG. 13 depicts a micro-machined filter assembly for CTC isolation according to an embodiment of the invention;

FIG. 14 depicts a process flow for the fabrication of etched silicon micro-machined filters according to an embodiment of the invention;

FIG. 15 depicts a process flow for the fabrication of embossed polymeric micro-machined filters according to an embodiment of the invention;

FIGS. 16A through 16C depict a process flow for the fabrication of etched silicon carbide micro-machined filters according to an embodiment of the invention;

FIG. 17 depicts an integrated micro-machined silicon carbide filter within a micro-fluidic structure with a silicon substrate incorporating CMOS electronics;

FIG. 18 depicts an optical micrograph of a fluorescent image of live membrane stained SK-BR-3 cells captured on a micro-machined filter functionalized with anti-HER2 antibodies according to an embodiment of the invention.

### DETAILED DESCRIPTION

The present invention is directed to micro-machined filters and more specifically high aspect ratio micro-machined filters for use in separation and isolation of circulating tumour cells.

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims.

FIG. 1 depicts first and second methods 100A and 100B respectively of CTC isolation according to the prior art. First method 100A exploits Ficoll-Paque™ to separate blood to its components wherein Ficoll-Paque™ is normally placed at the bottom of a conical tube, and blood is then slowly layered above it. After being centrifuged, layers will be visible in the conical tube, from top to bottom: plasma and other constituents, a layer of mono-nuclear cells called buffy coat (PBMC/MNC), Ficoll-Paque™, and erythrocytes & granulocytes which should be present in pellet form. This separation allows easy harvest of PBMC's. Disadvantages of the technique include red blood cell trapping (presence of erythrocytes & granulocytes), which may occur in the PBMC or Ficoll-Paque™ layer. Major blood clotting may sometimes occur in the PBMC layer. Ethylene diamine tetra-acetate (EDTA) and heparin are commonly used in conjunction with Ficoll-Paque™ to prevent clotting. Second method 100B depicts a similar technique without the proprietary Ficoll-Plaque™, albeit with reduced separation definition through the centrifuging of the blood sample with a graded sucrose solution such that particles are separated by density. First and second methods 100A and 100B being density gradient centrifugation isolate the mononucleocyte (MNC) fraction which includes CTCs. Subsequent removal of this section of the processed sample and immunohistochemical staining of cytokeratin to detect the CTC requires trained pathologist to examine samples and is accordingly time consuming and expensive with a maximum CTC recovery rate of typically 70%.

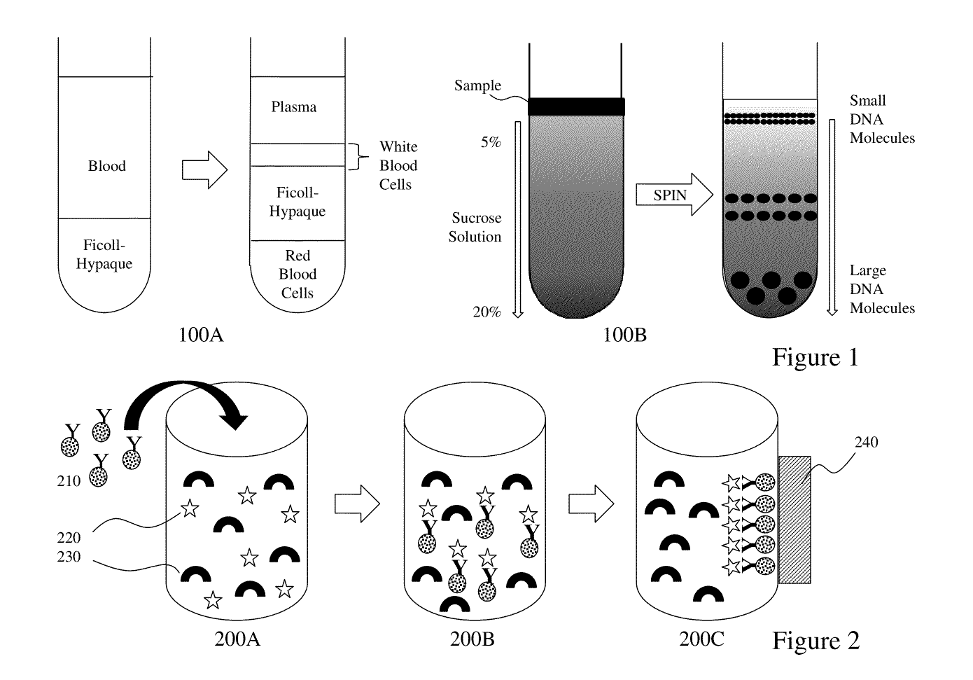

FIG. 2 depicts a method of CTC isolation by immunomagnetic separation according to the prior art which requires approximately 1 hour from sample collection to availability of results. The immunomagnetic separation (IMS) method is based upon the use of magnetic beads that are coated with antibodies specific to a particular protein or subsequence thereof that is expressed by a cell or bacteria, e.g. Escherichia coli (E. coli). This mixture is then agitated to increase the likelihood of the cell or bacteria binding to the antibody after which the cell-antibody-bead complex is separated from extraneous materials in the sample by use of a strong magnet such that these complexes are magnetically retained against the wall of the processing vessel during the removal of extraneous materials and the subsequent washing prior to further processing to concentrate the complexes for analysis. In the instance of bacterial cells this further processing may include cell wall rupturing by an enzymatic process to release adenosine triphosphate (ATP) which is measured with a microluminometer.

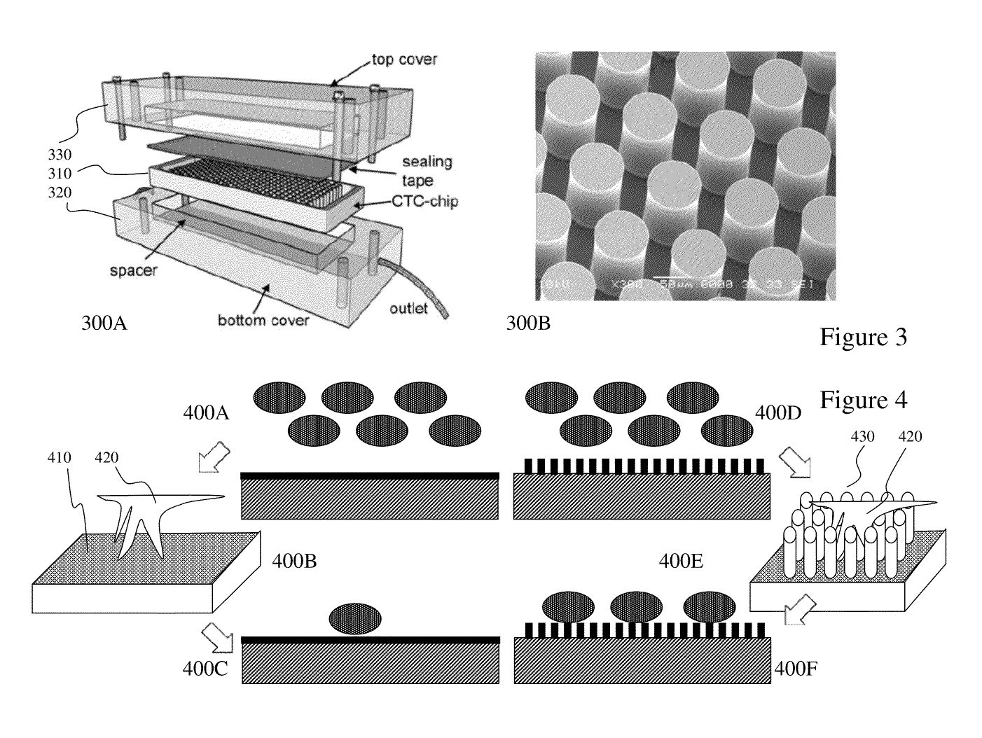

FIG. 3 depicts a method of CTC isolation through antibody coated surfaces according to the prior art of Nagrath as depicted by microfluidic system 300A and optical micrograph 300B which represents the subset of techniques for CTC isolation based upon molecular signature wherein an affinity based isolation using antibodies that bind to receptors specific to CTC cells derived from the epithelium, which are believed to be a main source for CTCs although this has not yet been demonstrated. The microfluidic system 300 consists of a microfluidic chip 310 etched in silicon, a manifold comprising lid 330 and base 320 to enclose the microfluidic chip 310, and a pneumatic pump (not shown) to establish flow. The dimensions of the chip microfluidic 310 reported by Nagrath were 25 mm×66 mm, with an active capture area of 19 mm×51 mm. As shown in optical micrograph it contains an array of microposts, 100 μm tall and 100 μm in diameter with an average 50 μm gap between microposts. For increased hydrodynamic efficiency, the repeated patterns of micropost arrays were shifted vertically by 50 μm for every row throughout the chip to maximize the interactions between micropost structures and cells. Overall this microfluidic chip 310 incorporates approximately 78,000 microposts fabricated with deep reactive ion etching (DRIE) within a surface area of 970 mm². The blood specimen collection and processing, macro-to-micro coupling, identification and enumeration of CTCs being common to other approaches within the prior art.

FIG. 4 depicts a method of CTC isolation through nano-patterned surfaces to enhance cell capture according to the prior art of Wang et al in “Three-Dimensional Nanostructured Substrates toward Efficient Capture of Circulating Tumor Cells” (Angew Chem Int Ed Engl. 2009; Vol 48(47), pp 8970-8973). Depicted by first to third schematics 400A through 400C is the prior art approach for EpCAM employing unstructured flat Si substrates coated with the adhesion promoting antibodies intended to capture the epithelial cells 420. Fourth to sixth schematics present the approach of Wang wherein 3D nanostructured substrates, specifically silicon-nanopillar (SiNP) arrays 430, allow for enhanced local topographic interactions between the SiNP substrates and nanoscale components of the cellular surface (e.g., microvilli and filopodia) of the epithelial cells 420. The rationale of Wang's approach was derived from studies by K. Fisher et al in “Biomimetic nanowire coatings for next generation adhesive drug delivery systems” (Nano Lett. 2009, Vol 9(2), pp 716-20) in which an enhanced adhesive force between a SiNP-coated bead and mucosal epithelial cells was attributed to local topographic interactions between SiNPs bound to the bead and nanoscale microvilli on the cell surfaces. At present enormous research efforts are being devoted to studying local topographic interactions between cells and a diversity of nanostructured substrates, which share nanoscale feature dimensions similar to those of cellular surface components and extracellular matrix (ECM) structures. However, most of this research has focused on achieving a better understanding of how nanostructures affect cellular behavior, for example, adhesion, viability, migration, differentiation, and morphology.

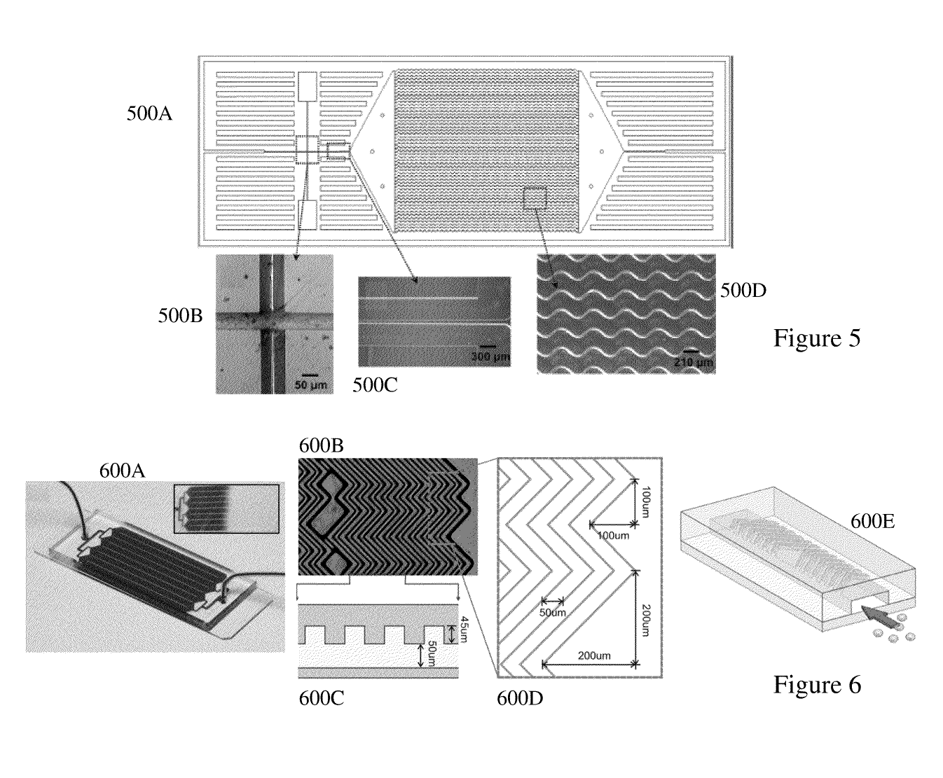

FIG. 5 depicts a method of CTC isolation through antibody coated surfaces with wavy channels to increase capture surface area according to the prior art of Adams et al in “Highly Efficient Circulating Tumor Cell Isolation from Whole Blood and Label-Free Enumeration Using Polymer-Based Microfluidics with an Integrated Conductivity Sensor” (J Am Chem. Soc., Vol. 130(27), pp 8633-8641). Adams reported a high throughput microsampling unit (HTMSU) formed from the transparent thermoplastic poly(methyl methacrylate (PMMA) which was selected due to its high fidelity of forming structures with high aspect ratio via microreplication, minimal nonspecific adsorption of whole blood components to its surface, and its ability to generate functional surface scaffolds through UV irradiation for the attachment of a variety of biological moieties.

First schematic 500A depicts a scaled diagram of the HTMSU showing the sinusoidally shaped capture channels with brightfield optical micrographs of the integrated conductivity sensor consisting of cylindrical Pt electrodes that were 75 μm in diameter with a 50 μm gap in first micrograph 500B and the single port exit where the HTMSU tapers from 100 μm wide to 50 μm while the depth tapers from 150 to 80 μm over a 2.5 mm region that ends 2.5 mm from the Pt electrodes in second micrograph 500C. Third micrograph 500D is a 5× magnification image of the sinusoidal cell capture channels. The intention being that microfluidic flow through the sinusoidal cell capture channels results in more cell-wall interactions and increased likelihood of bonding.

FIG. 6 depicts a method of CTC isolation through antibody coated surfaces with rapid microvortex flow according to the prior art of S. Stott et al in “Isolation of circulating tumor cells using a microvortex-generating herringbone-chip” (Proc. Nat. Acad. Sci., Vol. 107(43), pp 18392-1839′7). First schematic 600A depicts the herringbone (HB) device which consists of a microfluidic array of channels with a single inlet and exit wherein the inset shows the uniform blood flow through the device. First micrograph 600B depicts the grooved upper surface of the HB device which as shown in second schematic 600C have a profile height of 45 μm on the upper surface of the microfluidic channel and a minimum spacing between upper and lower surfaces of 50 μm. Second schematic 600D shows the dimensions of the herringbone pattern. The operating principle presented by Stott being that the herringbone structure in the upper surface of the microfluidic channel creates microvortices disrupting the laminar flow streamlines that cells travel, causing them to “shift” path, thereby increasing the number of cell-surface interactions in the antibody-coated device.

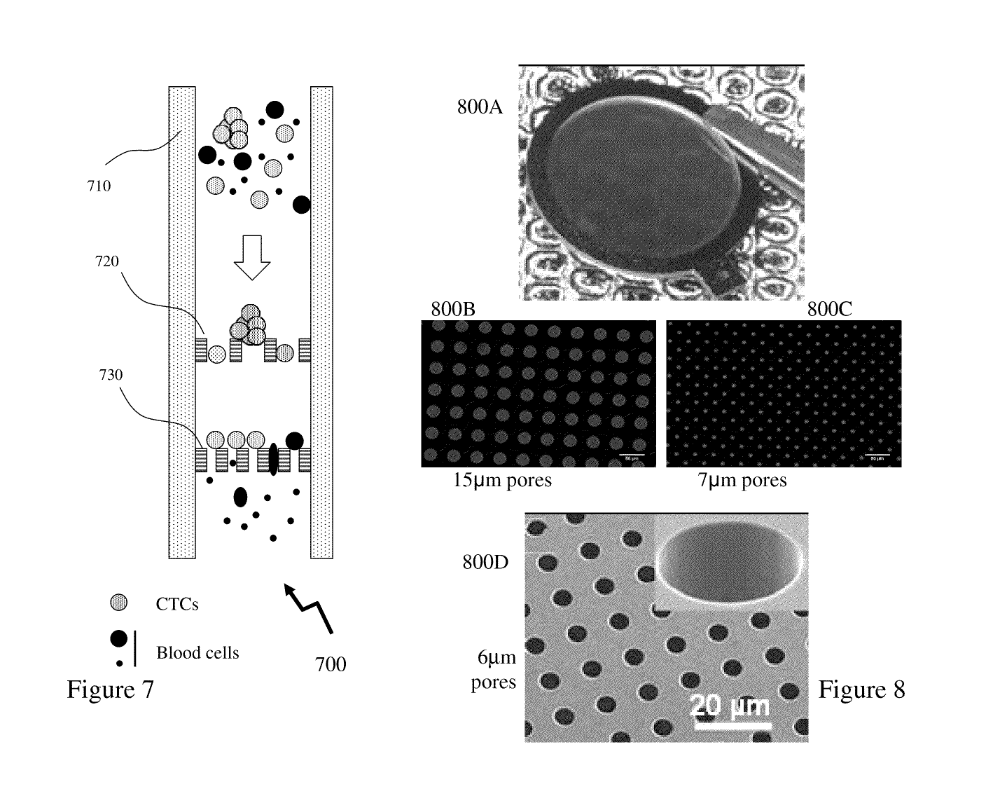

Referring to FIG. 7 there is depicted a CTC isolation methodology 700 according to an embodiment of the invention allowing multi-parameter isolation to increase specificity and sensitivity. The CTC isolation methodology 700 exploits as a first parameter mechanical filtration to isolate different cell populations by using reducing pore diameters in sequential stages, for example from 20 μm to 6 μm. However, as will be evident from discussion below in respect of the fabrication of the mechanical filters it is important for these to have high aspect ratio in some designs in order to exploit the differential deformability of blood components of similar dimensions as CTCs to pass through the filters whilst CTCs do not in the time period used. Accordingly the CTC isolation methodology 700 employs a mechanical assembly 710 to house multiple filters represented by first and second filters 720 and 730 respectively. As shown blood cells exhibit deformation in flow through capillaries, see for example U. Bagge et al in “Three-dimensional observations of red blood cell deformation in capillaries” (Blood Cells, Vol. 6(2), pp 231-9) unlike CTCs.

The second parameter exploited within the CTC isolation methodology 700 is specific antibody binding wherein the mechanical filters can be functionalized such that specific antibodies (Abs) bind to them via Ab regions not used for antigen recognition. For example the functionalization may be anti-human anti-human epidermal growth receptor 2 (HER2) Ab specific, where HER2 marker status is important in deciding targeted treatment, i.e. trastuzumab, in breast cancer or anti-EpCAM Ab specific for the isolation of EpCAM-positive cells. Other examples include Abs for specific stem cell markers, e.g. CD133, and Abs for hematopoietic-lineage marker as additional negative identification steps in respect of several diseases such as leukemia and lymphoma as well as hereditary blood disorders such as beta-thalessemia and sickle cell anemia

Referring to FIG. 8 there is depicted a micro-machined silicon filter 800A for CTC isolation according to an embodiment of the invention of diameter 10 mm with a 2 mm support ring and knob for handling. Symmetric patterns pores of dimensions 15 μm, 7 μm, and 6 μm are shown for different silicon filters 800A in first to third optical micrographs 800B through 800D respectively.

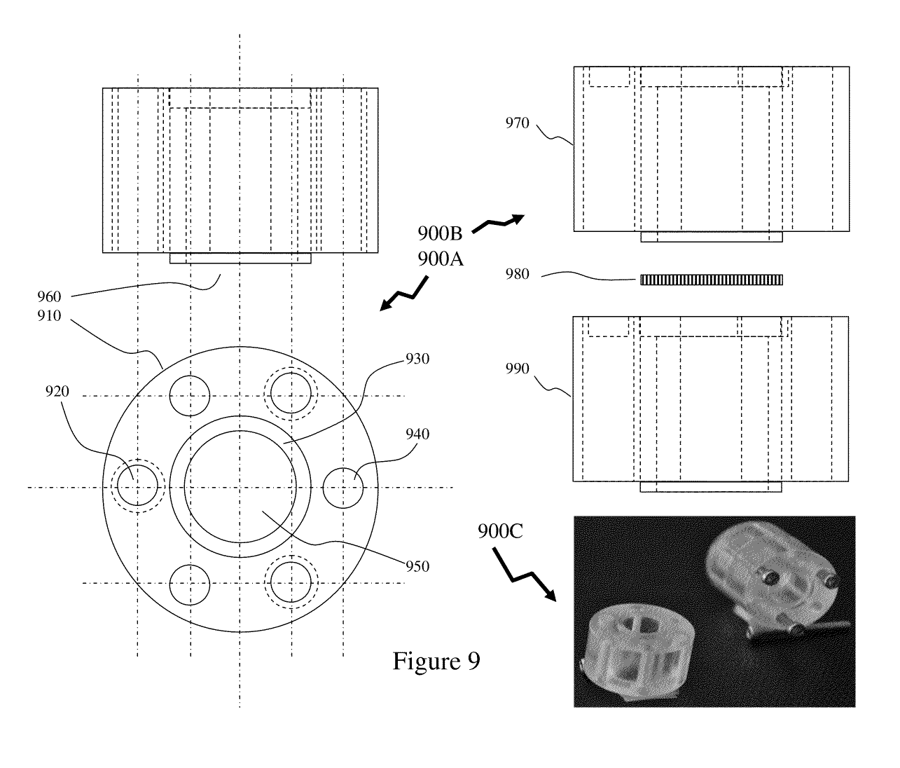

Now referring to FIG. 9 there is depicted a stackable modular filter element 900A allowing sequential filtering using functionalized and non-functionalized silicon filters such as depicted above in respect of silicon filter 800A in FIG. 8 above but without the handling knob. As depicted the modular filter element 900A comprises a central bore 950 for sample flow with a recess 930 at one end for the insertion of a silicon filter and a boss 960 at the other end for impinging on the silicon filter to hold it against the next modular filter element. First and second hole groups 920 and 940 respectively provide for bolt and guide rod insertion respectively. As depicted assembly 900B first and second modular filter elements 970 and 990 mount on either side of a silicon filter 980. Optical micrograph 900C shows an individual modular filter element and an assembled pair of modular filter elements.

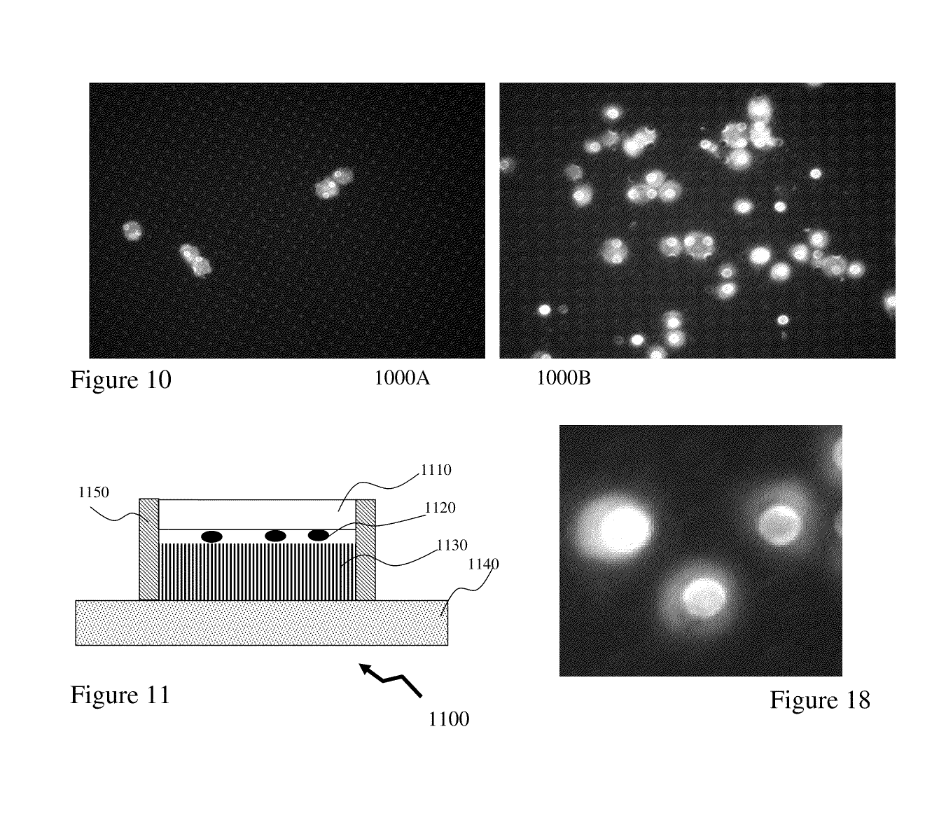

Referring to FIG. 10 there are depicted first and second optical micrographs 1000A and 1000B of Vybrant™ fluorescent dye-labelled MCF7 cells that have been filtered using micro-machined silicon filters according to an embodiment of the invention. Optical visualization of cells filtered using a micro-machined silicon filter according to an embodiment of the invention may be obtained as shown in FIG. 11 in assembly 1100 wherein a micro-machined silicon filter 1130 has been mounted upon a carrier 1140 and a transparent cover slip 1110 attached to fit within the filter handling ring 1150 of the micro-machined silicon filter 1130. Accordingly cells 1120 may be visualized directly on the filter, or alternatively may be removed for further processing by cell handling techniques within the prior art. Accordingly immunohistochemistry or immunofluorescence may be performed directly on the filter. In one approach to immunohistochemistry an antibody is conjugated to an enzyme, such as peroxidase, that can catalyse a colour-producing reaction whereas in immunofluorescence the antibody may also be tagged to a fluorophore, such as fluorescein or rhodamine.

Now referring to FIG. 12 depicts a stackable modular filter assembly 1200 for CTC isolation according to an embodiment of the invention. According there are shown stackable modular filter elements 1210, such as stackable modular filter element 900A presented above in respect of FIG. 9, that allow for the insertion of first to seventh filters 1220 through 1280 respectively such as micro-machined silicon filter 800A of FIG. 8 which according to the design of the stackable modular filter elements 1210 may be with or without the handling knob. First to seventh filters 1220 through 1280 being:

| | | |
|---|---|---|
| 1220 | Large pore filter | for filtering large CTC clumps |
| 1230 | Small pore filter | for filtering small CTC clumps |
| 1240 | Anti-CD45 filter | residual normal blood cells |
| 1250 | Anti-HER2 filter | HER2 + CTCs |
| 1260 | Anti-EGFR filter | EGFR + CTCs |
| 1270 | Anti-EpCAM filter | remaining EpCAM + CTCs |
| 1280 | Anti-CD133 filter | tumour stem cells |
| 1290 | Small pore filter | remaining CTCs |

Third to seventh filters 1240 through 1270 being functionalized micro-machined filters whereas first, second, eighth, and nine filters 1210, 1220, 1280 and 1290 respectively are non-functionalized micro-machined filters. Each functionalized filter being functionalized with the specific antibodies for CD45 (protein tyrosine phosphatase, receptor type C—PTPRC), HER2 (human epidermal growth factor receptor 2), EGFR (epidermal growth factor receptor), EpCAM (epithelial cell adhesion molecule), and CD133 (a glycoprotein also known in humans and rodents as Prominin 1 (PROM1) respectively for third to seventh filters 1240 through 1270.

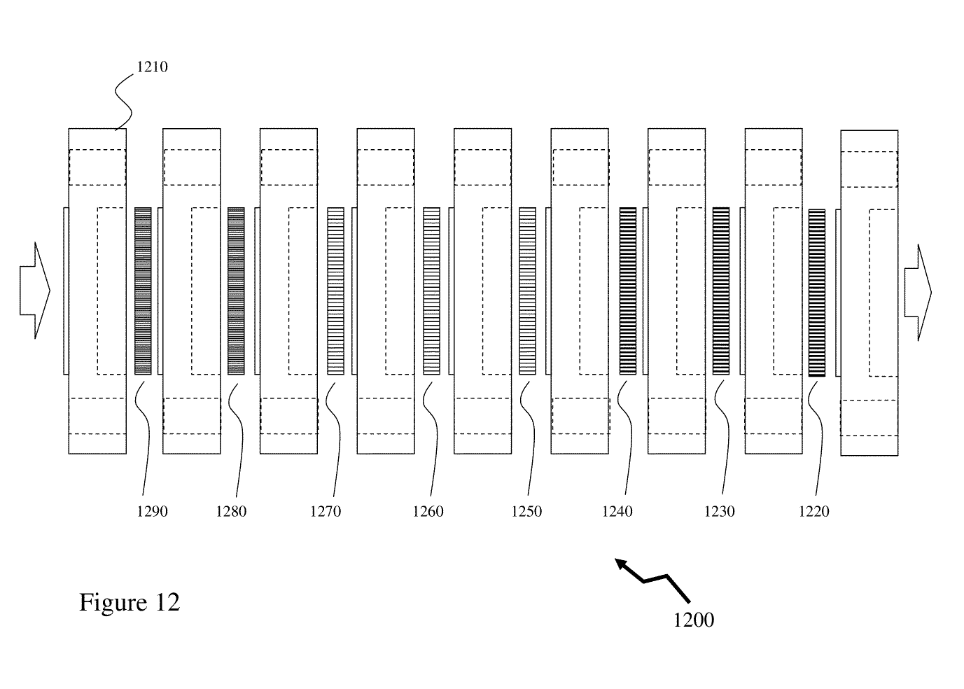

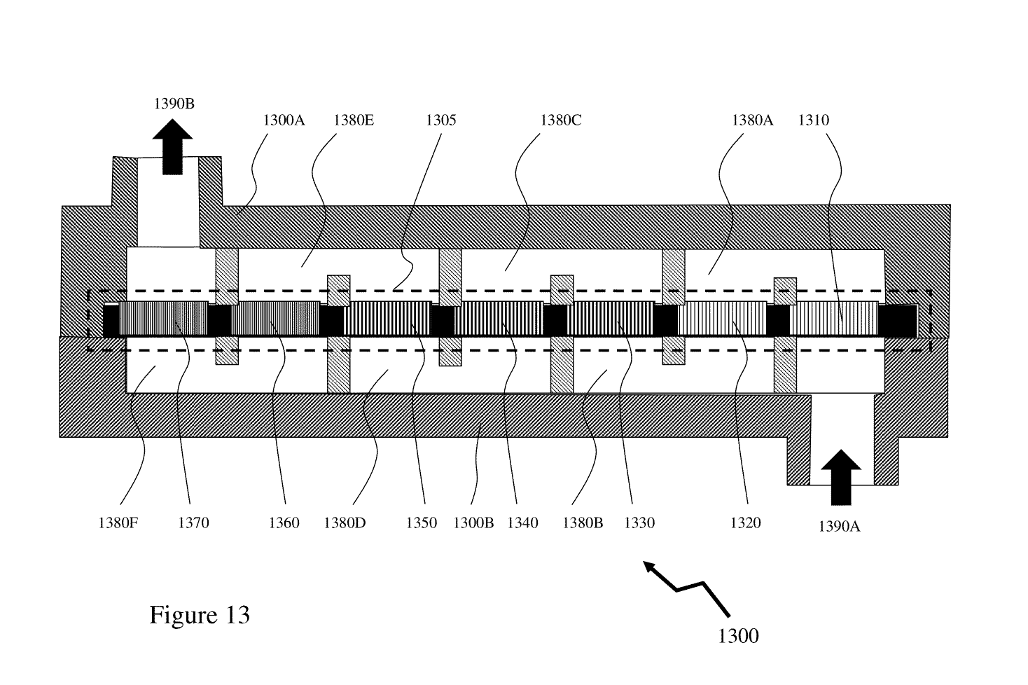

Referring to FIG. 13 there is depicted a micro-machined filter assembly 1300 for CTC isolation according to an embodiment of the invention wherein a micro-machined filter array 1305 is housed within a housing comprising a lower body 1300B with inlet 1390A and upper housing 1300A with outlet 1390B. When assembled the lower body 1300B and upper 1300A form a plurality of chambers 1380A through 1380F either side of the micro-machined filter array 1305 that has formed across it first to seventh filters 1310 through 1370 respectively wherein a sample entering from inlet 1390A and flowing to outlet 1390B is progressively filtered by the filters and flows from one filter to the other via the plurality of chambers 1380A through 1380F respectively. First to seventh filters 1310 through 1370 for example being second to eighth filters 1230 through 1290 respectively as presented above in respect of FIG. 12.

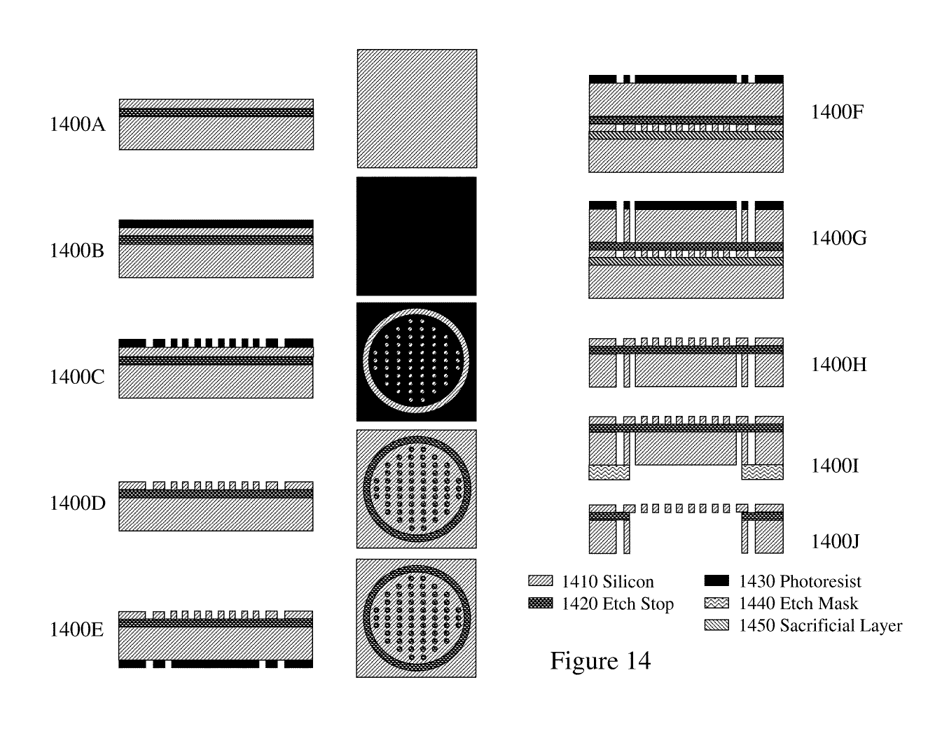

Accordingly, micro-machined filter array 1305 comprises multiple micro-machined filter elements of varying dimensions which may as evident from discussion below in respect of FIGS. 14 through 16 may be formed simultaneously in silicon, polymer, or silicon carbide respectively and selectively functionalized. It would also be evident that such a linear array of filters provides for reduced handling between the filtering process and visualization with a single micro-machined filter array 1305 replacing multiple discrete filters. It would be further evident to one skilled in the art that the micro-machined filter array 1305 may itself be arrayed to provide a single element containing multiple CTC filtering structures.

Referring to FIG. 14 there is depicted a process flow for the fabrication of etched silicon micro-machined filters according to an embodiment of the invention such as micro-machined silicon filter 800A, micro-machined silicon filter 1130, first to seventh filters 1220 through 1280, and first to seventh filters 1310 through 1370 in FIGS. 8, 11, 12, and 13 respectively. Accordingly the process begins with step 1400A wherein a layer of silicon 1410 is deposited above an etch stop 1420 upon a substrate. Subsequently in step 1400B a layer of photoresist 1430 is spin-coated onto the substrate and patterned in step 1400C to provide a circular opening and a plurality of openings within the photoresist 1430. The photoresist 1430 forming an etch mask for etching the silicon 1410 in step 1400D which is then removed and a second photoresist 1430 is spin-coated and patterned onto the backside of the substrate in step 1400E such that the rear-sided photoresist pattern is aligned to the pattern etched into the silicon 1410 in steps 1400B through 1400D.

Next in step 1400F a second substrate which has a sacrificial layer 1450 deposited upon it is attached such that the sacrificial layer 1450 and silicon 1410 are coupled. The second substrate is then coated with photoresist and patterned in step 1400F. The second substrate providing mechanical support for the backside etching of the substrate to the etch stop 1420 in step 1400G through the photoresist pattern formed in step 1400F. The second substrate is then removed through the sacrificial etching of the sacrificial layer 1450 in step 1400H. Next in step 14001 an etch mask is applied to the back side of the substrate which is etched in step 1400J together with the removal of the etch mask 1440 and etching of the etch stop 1420 thereby leaving a free standing micro-machined filter with a silicon ring support structure. It would be evident to one skilled in the art that the initial starting configuration in step 1400A may be varied according to the requirements of the micro-machined filter such that etch stop 1420 may for example be an insulator such that the initial wafer is therefore what is referred to as a silicon-on-insulator (SOI) wafer. Accordingly etch stop 1420 may for example be silicon oxide such that removal of the substrate is followed by an oxide etch process in step 1400J to release the micro-machined filter.

A limitation of the current silicon (Si) filters is their lack of transparency which limits the possibility of imaging cells in the pores or on the other side of the Si filter. Accordingly, the inventors using a process flow similar to described above in respect of FIG. 14 have developed a novel fabrication process for making micro-machined filters in transparent silicon dioxide. Using wafers with a PECVD SiO2 layer, the micro-machined filters are etched using deep reactive ion etching (DRIE) allowing etching of both the Si substrate and SiO2 etch stop layer in the same run. According to an embodiment of the invention approximately 300 μm thick silicon wafers with an approximate 2 μm SiO2 layer on the backside, and an approximate 10 μm SiO2 layer deposited by PECVD on the top are employed. Using photolithography holes are patterned into the top SiO2 layer and 1-2 mm wide rings (for handling) are formed in the substrate using RIE for the thin SiO2 and DRIE for the Si. Next, the wafer is flipped and attached to a handling wafer. The filter holes are then etched into the thick SiO2 by DRIE, and then the plug at the center of the ring released either by an isotropic dry etch, for example XeF2, or a wet etch, for example tetramethylammonium hydroxide (TMAH). The handling wafer is then detached, and the filters collected. For example using filters with a 7 mm diameter, 5 mm diameter filter and 1 mm ring, >300 filters can be accommodated in a single 6″ (150 mm) wafer with either constant or varying design parameters.

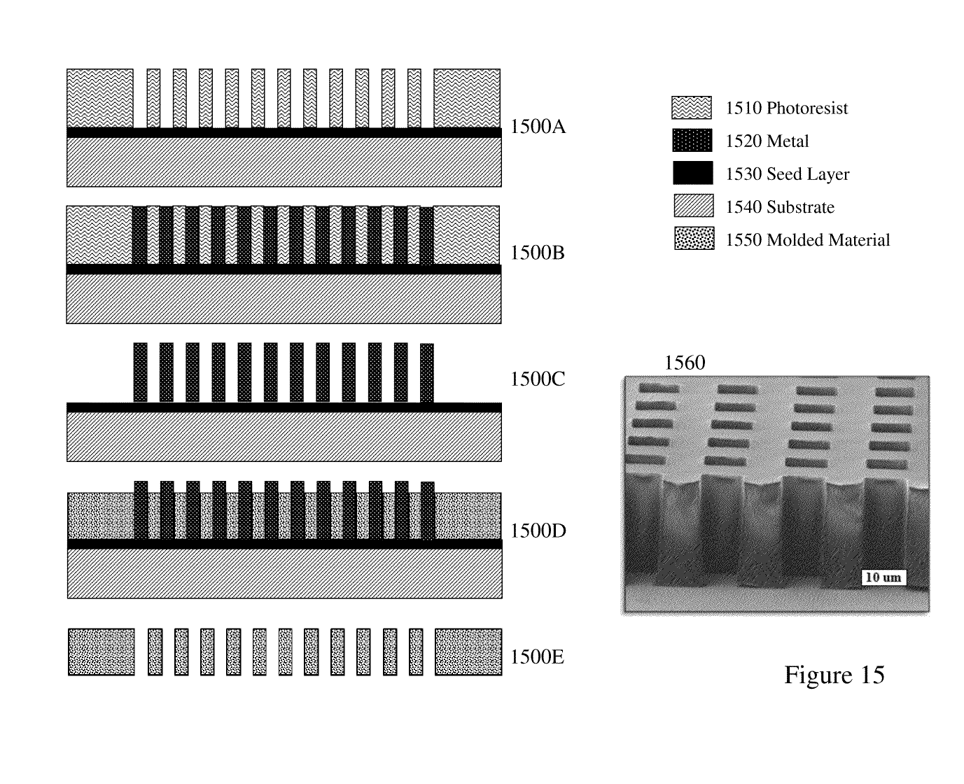

Now referring to FIG. 15 there is depicted a process flow for the fabrication of embossed polymeric micro-machined filters according to an embodiment of the invention through micromolding. Micromolding refers to fabrication of microstructures using molds to define the deposition of the structural layer. After the structural layer deposition, the final micro-fabricated components are realized when the mold is dissolved in a chemical etchant that does not attack the structural material. Micromolding is an additive process, in that the structural material is deposited only in those areas constituting the microdevice structure. In contrast, bulk and surface micromachining, such as described above in respect of the formation of a silicon micro-machined filter in FIG. 14, are examples of subtractive micromachining processes.

Micromolding describes a process that can be used for the manufacture of high-aspect-ratio, 3D microstructures in a wide variety of materials including metals, polymers, ceramics, and glasses. As shown in FIG. 15 according to an embodiment of the invention high-intensity, low-divergence, hard x rays are used as the exposure source for the lithography within a photoresist 1510, such as polymethylmethacrylate (PMMA). Thicknesses of several hundreds of microns and aspect ratios of more than 100 have been achieved within the prior art. A characteristic x-ray wavelength of 0.2 nm allows the transfer of a pattern from a high-contrast x-ray mask into a resist layer 1510 with a thickness of up to 1000 μm so that a resist relief may be generated with an extremely high depth-to-width ratio. The resist layer 1510 being formed upon a substrate 1540 with a sacrificial seed layer 1530. The openings in the patterned resist can be preferentially plated with metal 1520 in step 1500B, yielding a highly accurate complementary replica of the original resist pattern. The mold is then dissolved away in step 1500C to leave behind plated structures with sidewalls that are vertical and smooth. It is also possible to use the plated metal structures as an injection mold. Next in step 1500D a molding material 1550 is applied to the injection mold and cured. Next in step 1550E the metallic mold in the metal 1520 and seed layer 1530 are removed, leaving behind free-standing micro-replicas of the original pattern.

Conventionally, lithography requires a short-wavelength collimated x-ray source like a synchrotron which is expensive. Consequently, processes using conventional exposure sources are being developed with photoresists with high transparency and high viscosity can be used to achieve a single-coating mold thickness in the range of 15 μm to 500 μm. Thicker photoresist layers may be realized by multiple coatings. In such photoresist layers, standard ultraviolet (UV) photolithography is used to achieve mold features with aspect ratios exceeding 10:1. Photosensitive polyimides may also be used for fabricating the plating molds. The photolithography process is similar to conventional photolithography, except that polyimide works as a negative resist. All methods described above make use of lithography techniques to make a mold, but dry etching of polyimides to form high-aspect-ratio molds has also been reported within the prior art. In these methods, some modifications of traditional reactive ion etching (RIE) systems may be necessary to achieve high-aspect ratios including for example, dry etching, fluorinated polyimides, and a titanium (Ti) mask.

Optical micrograph 1560 in FIG. 15 depicts a fabricated high-aspect ratio polymeric filter. It would be evident that the larger thickness of high-aspect-ratio structures provides for greater stiffness perpendicular to the substrate. Plated nickel (Ni), copper (Cu), or alloys that containing these are examples of metallic masks, e.g. metal 1520, whilst chromium, silicon dioxide, polyimide, photoresist, and titanium are examples of the sacrificial material, seed layer 1530. In addition to soft polymers the process is compatible with hard polymers with excellent optical qualities such as cyclic olefin copolymers, which may have their surface chemistry modification with non-fouling coatings or with proteins for antibody binding. Other polymers may include thermoplastic materials such as poly(methylmethacrylate), polycarbonate

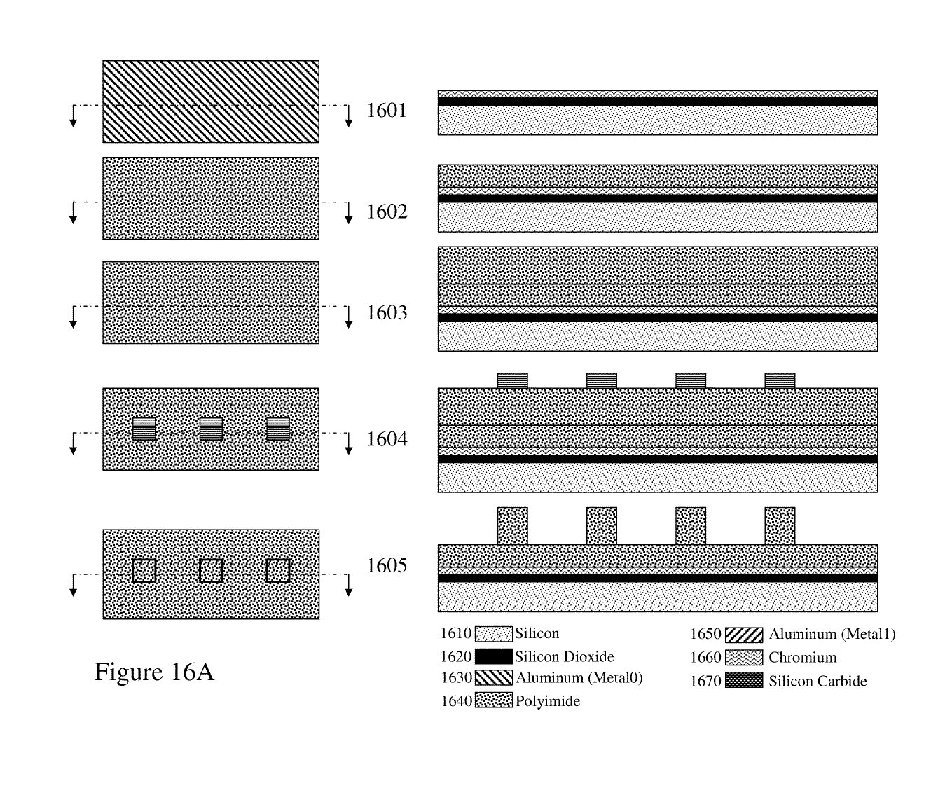

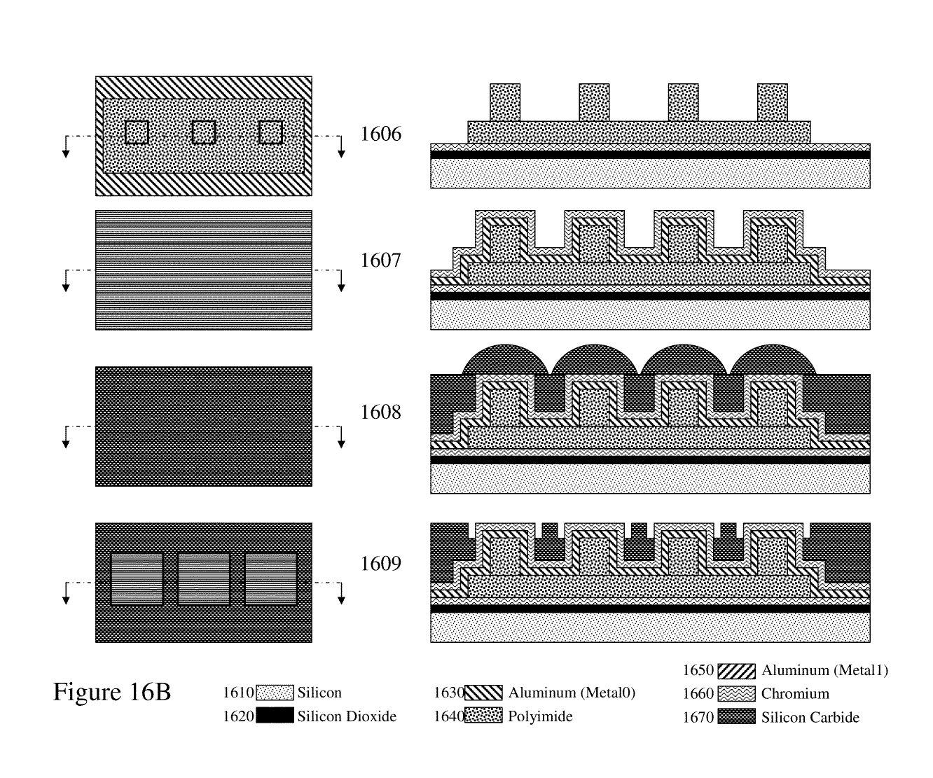

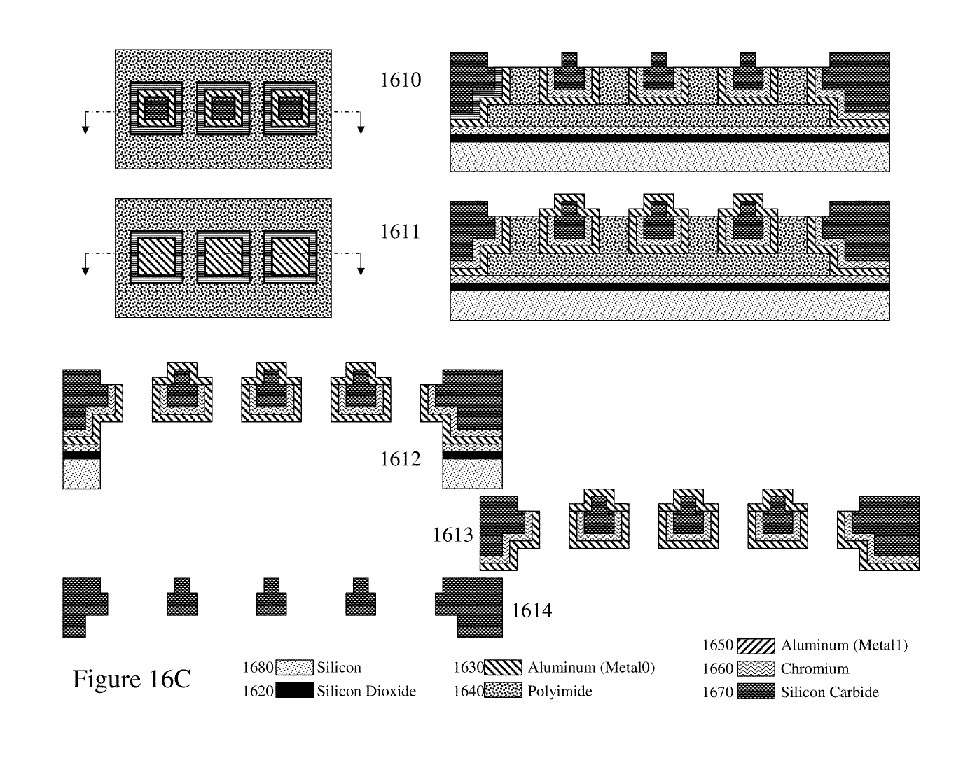

Referring to FIGS. 16A through 16C there is depicted a process flow for the fabrication of etched silicon carbide micro-machined filters according to an embodiment of the invention wherein the process steps are shown in plan and cross-sectional views. In first step 1601 a silicon wafer 1680 is provided, the silicon wafer 1680 which may contain CMOS electronics or it may not, and is coated with metallization, such as chromium 1660. Whilst shown as a blanket deposition in step 1601 this may be a deposition and patterning step such that the metallization provides an electrical interconnection pattern upon the surface to connect to the micro-machined structure in subsequent processing steps. In such instances an additional metal may be employed, such as aluminum (metal 0) 1630 with a chromium 1660 capping layer for reduced electrical resistance in the electrical connections.

Where the chromium 1660, or aluminum (metal 0) 1630 is employed, is directly deposited to patterned regions where the silicon wafer 1680 contains a processed CMOS substrate there would typically be present a passivation or planarization layer such as phosphosilicate glass, silicon oxide, or nitride. Optionally a 2.5 μm layer of silicon dioxide 1620 may be provided to reduce electrical feed-through from any electrical interconnects formed to the Si CMOS if implemented within the silicon wafer 1680. This layer may be applied prior to the metallization in step 1601. Next in step 1602 the metalized silicon wafer is coated with a 0.5 μm layer of polyimide 1640. The 0.5 μm polyimide layer 1640 being an easily removed sacrificial layer to release the structure as finally formed. On top of the sacrificial polyimide layer 1640 a further 2 μm spin-on polyimide layer is deposited in step 1603 and patterned in step 1604 by the deposition of an etch mask. The etch mask allowing the patterning of the 2 μm polyimide studs in step 1605 that will ultimately be removed to form the lateral gaps between the micro-machined elements. The etch mask may be a metal, such as chromium 1660, photoresist or another material providing the desired selectivity of etch between the polyimide and itself.

Now referring to FIG. 16B at step 1606 the initial 0.5 μm polyimide 1640 is patterned and etched to provide anchors for the micro-machined structures to the silicon wafer 1680 where this is desired. Next at step 1607 a 60 nm aluminum (metal 0) 1630 layer is deposited across the entire wafer surface forming the bottom and lateral structural interconnect, and the adhesion layer for the anchors, and is capped with an 160 nm chromium 1660 layer which will act as the etch stop for the silicon carbide 1670 structural layer. Accordingly in step 1608 a 2 μm silicon carbide (SiC) 1670 layer is deposited across the surface and in step 1609 is patterned leaving regions around the studs exposed. This region is then etched in step 1609 to expose the 60 nm chromium 1630/80 nm aluminum 1630 atop the 2 μm polyimide 1640 studs.

Now referring now to FIG. 16C there is shown the next step 1610 wherein these thin films atop the 2 μm polyimide 1640 studs are etched back sufficiently to expose the top of the polyimide 1640 studs. Accordingly at this point the elements of the micro-machined structure are isolated one from another as there is now no continuous SiC 1670 film bridging over the polyimide 1640 studs. In step 1611 the SiC 1670 is patterned with metallization for electrical interconnects, heaters, and other electrical structures according to the requirements of the micro-machined devices being fabricated. This metallization also allowing according to some embodiments of the invention for enhanced binding for functionalizing antibodies, provisioning of structures terminating in an antibody, the formation of nanostructures within the metal, and the integration of additional sensors with the CTC detection.

According to the particular design of the micro-machined structure as to whether it is to be integrated with the silicon wafer 1680, free-standing with metallization or free-standing then the process flow may be varied. In integrated substrate form 1612 the silicon wafer 1680 has been processed to provide an opening such that the SiC 1670 elements provide the required micro-machined filter with metallization provided on the filter surfaces. In first released form 1613 the silicon wafer 1680 has been removed through processing to provide a free-standing micro-machined SiC 1670 filter with metallization. In second released form 1614 all metallization etc has been removed leaving a free-standing micro-machined filter formed from SiC 1670. It would be evident to one skilled in the art that the depicted plan view schematics of the process flow presented in FIGS. 16A through 16C together with the cross-sectional views are intended to provide the reader with visualization of the process flow and are not intended to be representative of the structures that would be implemented to form micro-machined filters.

It would be evident to one skilled in the art that alternate process flows with either reduced complexity or increased complexity may be implemented to achieve the required micro-machined filters employing SiC 1670. Examples of other low temperature silicon carbide process flows allowing integration of filters, electronics, micro-fluidics, and functionalization include for example M. El-Gamal in “Low Temperature Ceramic Microelectromechanical Structures” (US Patent Application 2011/0,111,545), F. Nabki et al in “Low Temperature Ceramic Microelectromechanical Structures” (US Patent Application 2009/0,160,040) and M. El-Gamal et al in “Low Temperature Wafer Level Processing for MEMS Devices” (US Patent Application 2011/0,027,930).

Optionally the metallization deposited in step 1601 allowing the formation of electrical interconnects beneath the MEMS structure may be omitted. Alternatively the metallization used may be other than chromium according to the design requirements of the structure and performance requirements, other metallizations including for example aluminum (metal 0), gold (Au), titanium (Ti), platinum (Pt), and TiPtAu. Whilst the process flow presented in respect of FIGS. 16A through 16C provides for lateral gaps within the manufacture of low temperature SiC structural layers the formation of the polyimide 1640 studs requires that the etching of the polyimide be timed to remove the second polyimide 1640 layer everywhere except the studs. Hence, variations in polyimide 1640 quality can easily result in the timed etch removing a portion of the initial 0.5 um polyimide 1640 release layer. Optionally an alternative process flow may be implemented wherein an etch stop is provided between the two polyimide 1640 layers.

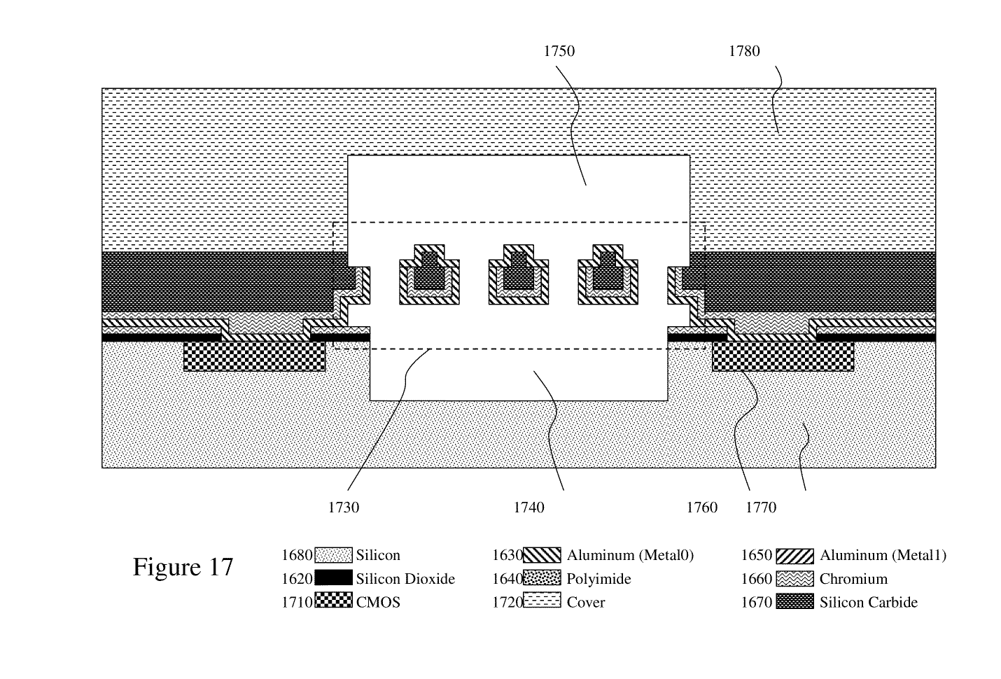

It would also be evident that the silicon wafer 1680 may have been pre-processed to include for example micro-fluidic structures that are etched into the surface prior to the formation of the micro-machined silicon or silicon carbide filter structures with or without attendant metallization. In such instances the silicon wafer 1680 may be packaged with a second processed silicon wafer with micro-fluidic structures to form an integrated assembly with direct electrical readout from the embedded EIS. Optionally, the second processed silicon wafer with micro-fluidic structures may be removed or be implemented in another material for removal for optical visualization. Alternatively a good quality transparent material of limited thickness may allow direct visualization. Such an option being depicted in FIG. 17 wherein a micro-machined SiC filter 1730 formed in a first substrate 1770 above a first micro-fluidic structure 1740 is covered with second substrate 1780 that has a second micro-fluidic structure 1750. Also formed within first substrate 1770 is CMOS electronics 1760.

It would also be evident that the integration of electrical structures within the micro-machined filters would allow for the use of techniques such as electrical impedance spectroscopy (EIS) for the automated measurement of captured cells at one or more filters either discretely or in combination with the optical visualization techniques. Accordingly, an EIS system may be implemented in CMOS and integrated into the silicon substrate within which the micro-machined filter devices are implemented, see for example V. Chodavarapu in “Self-Calibrating High-Throughput Integrated Impedance Spectrometer for Biological Applications” (US Patent Application 2011/0,115,499). It would be evident that such EIS integration may be implemented in other material systems for the micro-machined filters other than silicon carbide such as silicon and molded polymers. In the instance of molded polymer, such as described above metallization may be deposited and patterned prior to etching of the metal 1520 and seed layer 1530 to release the molded structure.

Chodavarapu also discloses an exemplary biochemical sensor for glucose although it would be evident that other binding elements may be used for cholesterol and specific blood ceils for example. In the instance of glucose the binding protein is glucokinase (GLK) which is attached to the gold metal electrodes of the sensor through a linker molecule. Accordingly as the GLK will only bind with the glucose wherein it will undergo a physiochemical change which results in a change in impedance for the electrodes to which it is attached. Accordingly, the more glucose present the higher the amount of glucose that will bind with the GLK protein and the greater the change in impedance measured with an EIS measurement system. For GLK Chodavarapu teaches that the linker molecule between the GLK and gold electrode was formed in four different steps including a self-assembly monolayer, melamine, nickel and glucose.

Using the processing techniques described above the inventors have manufactured micro-machined Si filters with 10 mm diameter and up to 542,833 holes per filter with varying hole diameters between 20 μm and 6 μm in 1 μm steps, and with varying opening ratios up to 50%. Such filters offer low flow resistance to the flow of sample fluids. Using such filters functionalized for MCF7, the inventors have used them for filtering MCF7 cells from 2 ml of solution in less than 2 min and were able to detach and culture the cells.

Within another embodiment of the invention anti-HER2 antibodies were attached covalently to 8 μm and 15 μm filters. Functionalized and non-functionalized filters were then used in a stackable modular filter assembly such as stackable modular filter assembly 1200 in FIG. 12 above to isolate SK-BR-3 cells which are known to overexpress HER2. 10,000 cells were spiked in 2 ml buffer and manually passed through the filters, and imaged as shown in FIG. 18. The inventor's interpretation is that these constitute cells squeezing through the pores and that the increased brightness overlaid with the pore is due to the fact that the transiting cells are imaged along their long axis, and the fluorescence is integrated over a large volume. This will need to be confirmed by confocal microscopy. The percentage of captured cells was deduced by counting the cells in the media that was flown through the filter as given by Table 1 below. These results indicate that for large holes affinity binding only modestly improves filtration, but for smaller holes, where cells are slowly squeezed through, it significantly increases the capture rate.

TABLE 1
Percentage of SKBR-3 cells retained by filter

| Coating | 15 μm Holes | 8 μm Holes |
|---|---|---|
| Anti-HER2 | 25% | 81% |
| Control | 20% | 19% |

The size and shape of the micro-machined filters impacts the selectivity of the filter towards different cells. Accordingly, in addition to hole size, such as the 20 μm to 6 μm exploited to date for making stacks of filters, varying hole geometries can be employed to isolate specific cell types. Due to the flexibility of the micro-fabrication processes described above in respect of silicon, polymer, silicon carbide with and without metallization various hole geometries may be employed including for example circular, elliptical, square, rectangular, tear drop, and star. Additionally, the deformation characteristics of cells vary so that flow rate and pressure may also be adjusted in targeting the isolation of specific cell types with micro-machined filters according to embodiments of the invention. Within individual stackable modular filter elements and the overall stackable modular filter assembly coatings may be applied, as would be evident to one skilled in the art for minimizing the binding of red blood cells so that raw blood may be processed directly. For example Teflon™ or Parylene™ may be treated with plasma and functionalized with non-fouling polyethylene glycol silanes, pluronics, or other inert coatings.

As described above the micro-fabricated filters are functionalized to capture part or the whole gamut of CTC cells. The functionalization being with one or more antibodies against particular cancer markers including, but not limited to EpCAM, EGFR (overexpressed in many cancers), HER2 (overexpressed in HER2+ breast cancers) chemokine receptors including CXCR4 and CCR7 (implicated in promoting metastasis to specific sites), the receptor tyrosine kinase Met (implicated in poor-outcome basal breast cancers), EMT markers such as E-cadherin, and stem cell markers such as CD44, CD29 and CD133. Stem cells are defined as CD44+CD24−, and thus the cells captured on the filters could be stained for CD24 to confirm that they are negative, or a filter for CD24+ cells could be added to the stackable modular filter assembly to eliminate these cells.

Once captured, CTC cells on the micro-fabricated filters may be further processed for “on-chip” immunostaining and immunohistochemistry (IHC)/immunofluorescence (IF). Typically, filters with immobilized cells will be stained with processes similar to conventional tissues slices, but with immunostaining protocols optimized for direct staining on the filters. IHC and IF can then be used to detect the markers used for isolation as well as non-cell-surface markers including ER and PR (for breast cancer subtype), ALDH1 (stem cell marker), as well as Vimentin, Twist, Slug and β-Catenin (EMT markers) to both confirm the specificity of isolation and to assess CTC heterogeneity, In addition, it would be evident that processing of the filters with autostainers may be performed in order to streamline and standardize the staining process. Similarly fluorescent imaging of filters may be performed automatically.

It would be evident to one skilled in the art that the stackable modular filter assembly may be implemented in different configurations to that shown above in respect of FIGS. 9, 12 and 13 as well as others to reduce dimensions, improve manufacturing processes, reduce costs as well as provide for locking/unlocking mechanisms, and improved assembly and disassembly procedures.

Within a stackable modular filter assembly the stacking order and filtration may be varied according to the cell types to be captured and the optimization of yield. Additionally other constraints may impact the construction and implementation of the stackable modular filter assembly including but not limited to the number of stains that can be identified simultaneously. However, it would be evident to one skilled in the art that a library of filters with an understanding of their efficiency in capturing cells with particular size, rigidity and surface markers for capturing CTCs, EMT-CTCs and CCSC can be developed as well as predetermined filter sequences. Through the use of semiconductor manufacturing methodologies the cost of micro-machined filters should be low.

It would be evident to one skilled in the art that even with only one or two affinity markers embodiments of the invention represent a low cost, quick technology for isolating cell clusters as protocols once established may be performed in a manner similar to other routine tests with cancer patients and high-risk patients for example with automated imaging systems for seamless clinical translation. It would be evident from the results above that the filtration of blood may be completed in a matter of minutes and that the stackable modular filter assembly is simple enough for analysis at the bedside.

It would be evident to one skilled in the art that whilst the preceding descriptions of embodiments of the invention in FIGS. 7 through 18 have been presented with respect to circulating tumour cells that other cell types may be specifically targeted or multiple sensors for materials within a fluid may be filtered and/or captured using embodiments of the invention. It would also be evident that whilst the descriptions consider the fluid to be blood drawn from a patient that the fluid may be continuously flowing through the filters for an extended period of time or that the fluid may be air or another gas or liquid such that embodiments of the invention may be deployed for multiple toxin detection or biochemical detection in conjunction with or in isolation with particular cell types.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps and means described above may be done in various ways. For example, these techniques, blocks, steps and means may be implemented in hardware, software, or a combination thereof. Physical implementations may be initially reduced to software form as computer instructions and/or data for transfer to computer automated manufacturing systems as well as for translation into other intermediate formats such as photolithography masks, direct write lithography sequences etc. Hardware implementations may combine processing units which may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above and/or a combination thereof with respect to the design of hardware implementations as well as characterization and analysis of results obtained from the use of the hardware implementations.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be rearranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages and/or any combination thereof. When implemented in software, firmware, middleware, scripting language and/or microcode, the program code or code segments to perform the necessary tasks may be stored in a machine readable medium, such as a storage medium. A code segment or machine-executable instruction may represent a procedure, a function, a subprogram, a program, a routine, a subroutine, a module, a software package, a script, a class, or any combination of instructions, data structures and/or program statements. A code segment may be coupled to another code segment or a hardware circuit by passing and/or receiving information, data, arguments, parameters and/or memory contents. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor and may vary in implementation where the memory is employed in storing software codes for subsequent execution to that when the memory is employed in executing the software codes. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels and/or various other mediums capable of storing, containing or carrying instruction(s) and/or data.

The methodologies described herein are, in one or more embodiments, performable by a machine which includes one or more processors that accept code segments containing instructions. For any of the methods described herein, when the instructions are executed by the machine, the machine performs the method. Any machine capable of executing a set of instructions (sequential or otherwise) that specify actions to be taken by that machine are included. Thus, a typical machine may be exemplified by a typical processing system that includes one or more processors. Each processor may include one or more of a CPU, a graphics-processing unit, and a programmable DSP unit. The processing system further may include a memory subsystem including main RAM and/or a static RAM, and/or ROM. A bus subsystem may be included for communicating between the components. If the processing system requires a display, such a display may be included, e.g., a liquid crystal display (LCD). If manual data entry is required, the processing system also includes an input device such as one or more of an alphanumeric input unit such as a keyboard, a pointing control device such as a mouse, and so forth.

The memory includes machine-readable code segments (e.g. software or software code) including instructions for performing, when executed by the processing system, one of more of the methods described herein. The software may reside entirely in the memory, or may also reside, completely or at least partially, within the RAM and/or within the processor during execution thereof by the computer system. Thus, the memory and the processor also constitute a system comprising machine-readable code.

In alternative embodiments, the machine operates as a standalone device or may be connected, e.g., networked to other machines, in a networked deployment, the machine may operate in the capacity of a server or a client machine in server-client network environment, or as a peer machine in a peer-to-peer or distributed network environment. The machine may be, for example, a computer, a server, a cluster of servers, a cluster of computers, a web appliance, a distributed computing environment, a cloud computing environment, or any machine capable of executing a set of instructions (sequential or otherwise) that specify actions to be taken by that machine. The term “machine” may also be taken to include any collection of machines that individually or jointly execute a set (or multiple sets) of instructions to perform any one or more of the methodologies discussed herein.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method comprising:
providing at least one micro-fabricated filter of a plurality of micro-fabricated filters, each micro-fabricated filter comprising holes of predetermined geometry and predetermined dimensions for capturing a predetermined cell type;
assembling the at least one micro-fabricated filter of the plurality of micro-fabricated filters within a housing;
exposing the at least one micro-fabricated filter of a plurality of micro-fabricated filters to a fluid;
measuring the at least one micro-fabricated filter of a plurality of micro-fabricated filters to determine the presence of the predetermined cell type.

2. The method according to claim 1 further comprising;
providing another micro-fabricated filter of the plurality of the micro-fabricated filters in conjunction with the at least one micro-fabricated filter of the plurality of micro-fabricated filters, wherein the other micro-fabricated filter of the plurality of micro-fabricated filters is intended to remove other cell types that may obfuscate the determination of the presence of the predetermined cell type.

3. The method according to claim 1 wherein,
providing the at least one micro-fabricated filter of the plurality of micro-fabricated filters comprises providing at least one of a metallic coating, nanostructures, and an antibody binding agent over a predetermined portion of the micro-fabricated filter.

4. The method according to claim 1 wherein,
the at least one micro-fabricated filter of the plurality of micro-fabricated filters comprises a matrix of holes formed within at least one of silicon, silicon dioxide, silicon carbide and a polymer.

5. The method according to claim 1 wherein,
providing the at least one micro-fabricated filter of the plurality of micro-fabricated filters comprises providing an antibody binding agent to enhance the capture of the predetermined cell type.

6. The method according to claim 1 wherein,
measuring the at least one micro-fabricated filter of the plurality of micro-fabricated filters to determine the presence of the predetermined cell type comprises at least one of optically inspecting and electrically measuring the at least one micro-fabricated filter of a plurality of micro-fabricated filters at least one of without further processing and with at least one of immunostaining, immunohistochemistry, and immunofluorescence.

7. A method comprising:
providing a series of micro-fabricated filters representing a predetermined subset of a plurality of micro-fabricated filters, each micro-fabricated filter comprising holes of predetermined geometry and predetermined dimensions for capturing a predetermined cell type; and
assembling the series of micro-fabricated filters in a predetermined order based upon the plurality of predetermined cell types.

8. The method according to claim 7 further comprising;
assembling the series of micro-fabricated filters in a predetermined order based upon the provisioning of functionalizations for capturing predetermined cell types to a predetermined subset of the series of micro-fabricated filters.

9. The method according to claim 7 wherein,
the predetermined order is intended to remove other cell types that may obfuscate the determination of the presence of a predetermined cell type upon the associated micro-fabricated filter.

10. The method according to claim 7 wherein,
providing at least one micro-fabricated filter of the series of micro-fabricated filters comprises providing at least one of a metallic coating, nanostructures, and an antibody binding agent over a predetermined portion of the micro-fabricated filter.

11. The method according to claim 7 wherein,
providing at least one micro-fabricated filter of the series of micro-fabricated filters comprises a matrix of holes formed within at least one of silicon, silicon dioxide, silicon carbide and a polymer.

12. The method according to claim 7 wherein,
providing at least one micro-fabricated filter of the series of micro-fabricated filters comprises providing an antibody binding agent to enhance the capture of the predetermined cell type.

13. The method according to claim 7 further comprising;
measuring at least one micro-fabricated filter of the series of micro-fabricated filters after exposing the series of micro-fabricated filters to fluid to determine the presence of the predetermined cell type comprises at least one of optically inspecting and electrically measuring the at least one micro-fabricated filter of a plurality of micro-fabricated filters at least one of without further processing and with at least one of immunostaining, immunohistochemistry, and immunofluorescence.

14. A device comprising:
a series of micro-fabricated filters representing a predetermined subset of a plurality of micro-fabricated filters, each micro-fabricated filter comprising holes of predetermined geometry and predetermined dimensions for capturing a predetermined cell type, wherein
the series of micro-fabricated filters are disposed in a predetermined order for sequentially filtering a fluid passing through them based upon the plurality of predetermined cell types.

15. The device according to claim 14 wherein,
the predetermined order is further based upon at least one of:
the provisioning of functionalizations for capturing predetermined cell types to a predetermined subset of the series of micro-fabricated filters; and
removing other cell types that may obfuscate the determination of the presence of a predetermined cell type upon the associated micro-fabricated filter.

16. The device according to claim 14 wherein,
providing at least one micro-fabricated filter of the series of micro-fabricated filters comprises:
providing at least one of a metallic coating, nanostructures, and an antibody binding agent over a predetermined portion of the micro-fabricated filter; and
providing a matrix of holes formed within at least one of silicon, silicon dioxide, silicon carbide and a polymer.

17. The device according to claim 14 wherein,
providing at least one micro-fabricated filter of the series of micro-fabricated filters comprises providing an antibody binding agent to enhance the capture of the predetermined cell type.

18. The device according to claim 14 further comprising;
a housing comprising:
an inlet port;
an outlet port;
a first portion for mounting the series of micro-fabricated filters to in predetermined locations relative to the inlet port and outlet port;
a second portion for demountably attaching to the first portion; wherein
the housing holds each micro-fabricated filter in place and prevents fluid flowing through the series of micro-fabricated filters from reaching any micro-fabricated filter without passing through the preceding micro-fabricated filters in the predetermined order.

19. The device according to claim 14 wherein,
a predetermined set of the series of micro-fabricated filters are fabricated in different positions within the same structure.

20. The device according to claim 14 wherein,
a predetermined set of the series of micro-fabricated filters are fabricated in different positions within the same structure and the structure allows inspection of the series of micro-fabricated filters with automated equipment.
